(19) United States
(12) Patent Application Publication
(10) Pub. No.: US 2022/0241181 A1
(43) Pub. Date: Aug. 4, 2022

(54) NOVEL COSMETIC AND DERMATOLOGICAL USES OF AN EXTRACT OF CISTUS MONSPELIENSIS

(71) Applicant: BASF Beauty Care Solutions France SAS, Lyon (FR)

(72) Inventors: Vincent BARDEY, Essey-les-Nancy (FR); Louis DANOUX, Essey-les-Nancy (FR); Florence HENRY, Essey-les-Nancy (FR); Solene MINE GENSOLLEN, Essey-les-Nancy (FR); Sabine PAIN, Lyon (FR); Nicolas PELLETIER, Lyon (FR)

(21) Appl. No.: 17/416,049

(22) PCT Filed: Dec. 10, 2019
(86) PCT No.: PCT/FR2019/052991

§ 371 Date: Mar. 1, 2022

(30) Foreign Application Priority Data
Dec. 21, 2018 (FR) 1873817

(51) Int. Cl. A61K 8/9789; A61Q 19/08; A61Q 19/02; A61K 8/37; A61K 8/49; A61K 36/185; A61P 17/00

(52) U.S. Cl. CPC A61K 8/9789; A61Q 19/08; A61Q 19/02; A61P 17/00; A61K 8/4966; A61K 36/185; A61K 8/37

(57) ABSTRACT

Novel cosmetic and dermatological uses of an extract of Cistus monspeliensis The invention relates to the non-therapeutic cosmetic use of an extract of Cistus monspeliensis for increasing and/or maintaining tissue homeostasis in the skin and/or the mucous membranes and/or the skin appendages, by increasing and/or maintaining the barrier function and/or cell differentiation in the skin and/or the mucous membranes and/or the skin appendages. Another subject relates to a non-therapeutic cosmetic care process notably comprising the topical application of the extract of C. monspeliensis or of a cosmetic composition comprising same. Yet another subject relates to the extract of C. monspeliensis, for its use for preventing and/or treating diseases induced by a decrease in the gene and/or protein expression of ECM1, advantageously lichen sclerosus or lipoid proteinosis, and/or for preventing and/or treating scars.

### TECHNICAL FIELD

The present invention relates to the non-therapeutic cosmetic or dermatological use of an extract of the plant Cistus monspeliensis for increasing or maintaining the tissue homeostasis of the skin and/or the mucous membranes and/or the skin appendages. The skin is an organ for protecting against external stressors such as stress, pollution, UV radiation and temperature changes. It acts as a barrier.

Correct homeostatic functioning of the skin and its capacity for regeneration are key processes in the defence and resilience capacity of the skin against external environmental stressors. Functioning defects or loss of cutaneous equilibrium and of its renewal capacity lead to the skin being less healthy.

Involucrin is a soluble protein synthesized in the keratinocytes, which permits maintenance of the cornified layer of the skin by participating in epidermal differentiation and, consequently, by facilitating the cohesion of the cornified layer. This is referred to as the barrier effect.

The ECM1 (Extracellular Matrix Protein 1) protein is an 85 kDa secreted glycoprotein which plays a key role in the structure and homeostasis of the skin. It participates in the formation and reconstitution of the basal membrane, the differentiation of dermal and epidermal tissues during development and postpartum. The study of skin pathologies associated with mutations resulting in a loss of function of the gene coding for ECM1 has highlighted its role in human beings. It is expressed in the skin, and in the epidermal keratinocytes.

ECM1 is situated in the basal layer of the epidermis, in dermal vessels and in the outer root sheath (ORS), but also in the suprabasal layers of the epidermis.

The ECM1 protein is also expressed in the dermis, where it binds to a great number of structural proteins such as perlecan, thus taking part in the organization of the basal membranes and in the assembly of collagen fibres.

The expression of ECM1 is downregulated in elderly skin and, conversely, is overexpressed in skin subjected to UV radiation. Patients having an ECM1 deficiency and exposed to sunlight have skin with scars and a more severe photo-aged appearance, compared with non-exposed skin. The presence of thick and persistent scars in pathologies linked to an ECM1 deficiency suggests an important role of this protein in tissue repair, remodelling or regenerating phases.

The ECM1 protein consequently represents a prime target in the fields of cosmetics and dermatology, which are in constant need of alternative ingredients that are notably active on the extracellular matrix (ECM) components, active in the formation of the matrix network, cell adhesion, and the regulation of tissue differentiation.

The fundamental involvement of this protein in tissue homeostasis makes it a favoured target for developing active ingredients for the skin, the scalp and its appendages (head hair, bodily hair, nails).

The plant C. monspeliensis, also known as Montpellier Cistus, is a plant commonly found in France, North Africa, and more generally around the Mediterranean basin. It is a fragrant shrub known to rapidly colonize arid and damaged soils, particularly after fires. Its essential oil is known for its anti-haemorrhagic and anti-infectious properties. The Applicant has discovered, surprisingly, that an extract of C. monspeliensis has the capacity for increasing the expression of both the ECM1 protein and the protein involucrin. This extract thus has an effect on tissue homeostasis and on the barrier function of the skin and the skin appendages.

One advantage of the extract according to the invention is that it is a topically acceptable active ingredient, and does not generate any red patches or allergies after application to the skin and/or mucous membranes.

Another advantage of the extract according to the invention is that it is easy to produce it on an industrial scale.

### PRIOR ART

The use of the plant C. monspeliensis in cosmetic compositions is already known. Patent application GB2443388 thus describes cosmetic compositions comprising a combination of alginate and of an extract of a plant chosen from Lavandula stoechas, Helichrysum italicum and C. monspeliensis regarding their use for combating acne. However, said document does not indicate the part of the plant from which the extract is obtained. Furthermore, the extract is obtained using a special process, vacuum microwave hydrodistillation. Finally, the extract is never used alone, but always in combination with an alginate.

Patent application FR2856299 describes several species of the Cistus genus comprising labdanic compounds and the effects thereof on promoting collagen synthesis. The extracts may thus be used for combating wrinkles and skin sagging.

Patent application FR2819718 describes several plant lipid extracts, including that of C. monspeliensis, and the use thereof in cosmetic products for combating the effects of ageing. However, said application does not describe an extract of the aerial parts of C. monspeliensis, and the lipid extracts are obtained by extraction with supercritical CO2.

Patent application JP 2011/162504 describes an extract of C. monspeliensis which stimulates ATP synthesis, for promoting cell growth and cell division. However, the use of an extract of the aerial parts of C. monspeliensis for increasing cell differentiation is not disclosed anywhere in said document, and all the less so is an extract of the aerial parts of the plant using water as sole solvent.

Patent application JP 2003/267865 discloses labdanic molecules present in the plant C. monspeliensis and the use thereof as a cell activator. However, the present invention is distinguished from this prior art in that the aim of the present invention is not the use of an extract of C. monspeliensis for increasing cell growth, but rather for increasing cell differentiation, making it possible to increase tissue homeostasis.

Thus, to the Applicant's knowledge, no prior art document discloses an extract of C. monspeliensis, and even less so an extract of the aerial parts of the plant, for maintaining and/or increasing the barrier function of the skin and/or of the skin appendages and/or the cell differentiation of the skin and/or the mucous membranes and/or the skin appendages. Neither does any prior art disclose the cosmetic use of said extract for maintaining and/or increasing tissue homeostasis or tissue renewal.

### DESCRIPTION OF THE INVENTION

A first subject of the invention thus relates to the non-therapeutic cosmetic use of an extract of C. monspeliensis for maintaining and/or increasing tissue homeostasis in the skin and/or the mucous membranes and/or the skin appendages.

For the purposes of the invention, the term “cosmetic use” means a non-therapeutic use, i.e. a use which does not necessitate a therapeutic treatment and which is intended for skin and/or mucous membranes and/or skin appendages that are healthy, i.e. which do not show any dermatological pathologies, and do not show any infections, inflammations, scars, diseases or skin complaints such as candidiasis, impetigo, psoriasis, eczema, acne or dermatitis, or couperosis, or wounds or injuries, or do not suffer from lichen sclerosus or lipoid proteinosis.

The extract according to the invention is a topically acceptable active extract. The term “topically acceptable” means herein a non-toxic extract that is suitable for topical application, which is non-irritant to the skin and/or the mucous membranes and/or the skin appendages, which does not induce an allergic response, and which is not chemically unstable.

The use of the extract according to the invention may be by oral or topical administration. Advantageously, the extract is applied topically. The term “topically” means the direct local application and/or spraying of the extract onto the surface of the skin and/or the mucous membranes and/or the skin appendages.

The term “mucous membrane” means the vaginal mucous membrane, the urogenital mucous membrane, the anal mucous membrane, the nasal mucous membrane, the oral mucous membrane, the ocular mucous membrane, the labial and/or gingival mucous membrane, preferentially the labial mucous membrane.

For the purposes of the invention, the skin includes the scalp. The term “skin appendages” means head hair, bodily hair or the nails, and is preferentially head hair. The extract according to the invention may be applied topically to all or part of the face and/or the body and/or the scalp and/or the skin appendages and/or the mucous membranes, chosen from the legs, the hands, the thighs, the stomach, the neckline, the neck, the arms, the torso, the back, the hair, the face, notably the area around the eyes, advantageously the neckline and/or the face and notably the area around the eyes, more advantageously the area around the eyes.

For the purposes of the invention, the term “tissue homeostasis” means maintaining the balance between the exchange and synthesis functions of the various components in the skin and/or the mucous membranes and/or the skin appendages, advantageously in the epidermis, in order to keep constant the biological parameters of the skin and/or the mucous membranes and/or the skin appendages constant when faced with modifications of the external environment.

Tissue homeostasis will be increased and/or maintained in the skin and/or the mucous membranes and/or the skin appendages from the moment when the cell differentiation in the skin and/or the mucous membranes and/or the skin appendages, and/or the barrier function of the skin and/or of the skin appendages is increased and/or maintained.

Thus, in one embodiment of the invention, the extract of C. monspeliensis according to the invention maintains and/or increases cell differentiation in the skin and/or the mucous membranes and/or the skin appendages. The term “increase and/or maintain cell differentiation” means increasing the gene and/or protein expression of ECM1 in the skin and/or the mucous membranes and/or the skin appendages.

Thus, the extract of C. monspeliensis is present in an amount which is effective for increasing cell differentiation when the increase in the gene and/or protein expression of ECM1 is of at least 30%, preferentially at least 40% in the presence of the extract of C. monspeliensis, in comparison with the level of gene and/or protein expression detected in the absence of the extract. In an advantageous embodiment of the invention, the increase in the expression of ECM1 is measured in “normal”, i.e. non-pathological, human keratinocytes, more advantageously in the presence of the extract of C. monspeliensis prepared as described in Example 1a). Very advantageously, it will be an increase in ECM1 gene expression measured by quantitative PCR (Q-PCR), under the conditions as described in Example 2a). Alternatively, it may be an increase in ECM1 protein expression measured by Western blotting, under the conditions described in Example 2b).

For the purposes of the invention, cell differentiation is distinct from cell growth or cell proliferation. Thus, in a very advantageous embodiment, the non-therapeutic cosmetic use of the extract of C. monspeliensis according to the invention is not intended for maintaining and/or increasing cell growth or cell proliferation. The extract of C. monspeliensis according to the invention is not intended either for increasing cell division.

In a particular embodiment, the extract is not intended either for increasing and/or maintaining the synthesis of ATP.

The term “increasing the barrier function” of the skin and/or of the skin appendages means increasing the thickness of the epidermis and/or promoting the cohesion of the cornified layer of the skin and/or the skin appendages. The extract of C. monspeliensis according to the invention is thus useful for limiting water losses in the skin and/or the skin appendages and/or for preventing the dehydration thereof.

Advantageously, the term “increasing the barrier function” means increasing the gene and/or protein expression of involucrin. Thus, the extract of C. monspeliensis is in an amount that is effective for increasing the barrier function when the increase in gene and/or protein expression of involucrin is of at least 50%, advantageously at least 100%, very advantageously at least 150%, in the presence of the extract of C. monspeliensis according to the invention, in comparison with the level of involucrin expression detected without any extract. In an advantageous embodiment of the invention, it will be an increase in the protein expression of involucrin and, even more advantageously, this increase will be measured in “normal”, i.e. non-pathological, human keratinocytes, more preferentially in the presence of the extract of C. monspeliensis prepared as described in Example 1a). In a particularly advantageous manner, this measurement of the increase in the involucrin protein expression will be performed by means of the ELISA technique, under the conditions described in Example 3.

The term “increasing the gene expression” means increasing the expression of mRNAs coding for ECM1 and/or involucrin. The term “increasing the protein expression” means increasing the protein synthesis.

In a particular manner, the extract of C. monspeliensis according to the invention increases and/or maintains tissue renewal in the skin and/or the mucous membranes and/or the skin appendages.

In one embodiment of the invention, the tissue renewal may be evaluated in vivo. Thus, the extract of C. monspeliensis according to the invention is considered as an amount effective for “increasing tissue renewal” when a reduction of at least 2, preferentially at least 3, of the number of days necessary for the complete renewal of the epidermis will be observed in the presence of the extract according to the invention, in comparison with the number of days required to renew the epidermis without any extract. Advantageously, this number of days is determined after the application, twice a day over a total period of 28 days, of a cream comprising the extract of C. monspeliensis on the forearms of a population of women from 45 to 65 years old, in comparison with a placebo cream without any extract. More advantageously, the number of days necessary for the epidermal renewal is measured by means of the DHA (dihydroacetone) method which consists in colouring the skin surface of interest using said DHA, which reacts with the epidermal proteins to form brown melanoid pigments. The time required for decolourization of the brown pigments corresponds to a renewal time considered as total. Very advantageously, the cream used comprises a final concentration of extract of C. monspeliensis prepared according to Example 1a) of 0.1% by weight relative to the total weight of said cream, as described in Example 4 of the present patent application.

Thus, the extract of C. monspeliensis may be used in the context of the invention for improving the surface appearance of the skin and/or the skin appendages and/or the mucous membranes. The term “improving the surface appearance” means improving the texture of the skin and/or the mucous membranes and/or the skin appendages. The extract of C. monspeliensis may moreover be used for increasing the radiance of the skin complexion. The term “increasing the radiance of the complexion” means increasing the luminous appearance of the skin and/or the homogeneity of the complexion and/or improving the radiance and/or reducing the dull and/or sallow complexion of the skin. In a particular embodiment of the invention, the extract of C. monspeliensis is considered as being in an amount effective for “increasing the radiance of the complexion” when the extract reduces the overall melanin content by at least 40%, advantageously at least 55% and more advantageously at least 60%. In a particularly advantageous embodiment, it is a measurement performed in B16 melanocytes cultivated in the presence of an extract of C. monspeliensis at a final concentration of 0.01% by weight relative to the final volume of the melanocyte culture. Very advantageously, it is the extract of C. monspeliensis as prepared according to Example 1a), under the conditions described in Example 6.

Finally, the extract of C. monspeliensis is useful for reducing pigmentation marks of the skin, participating in increasing the homogeneity of the complexion. Thus, the extract is considered as being in an amount effective for reducing pigmentation marks when it reduces the overall melanin content by at least 40%, advantageously at least 55% and more advantageously at least 60%. In an advantageous embodiment, it is a measurement performed in B16 melanocytes cultivated in the presence of an extract of C. monspeliensis at a final concentration of 0.01% by weight relative to the final volume of the melanocyte culture. Very advantageously, it is the extract of C. monspeliensis as prepared according to Example 1a), under the conditions described in Example 6.

In a particular embodiment of the invention, the extract of C. monspeliensis according to the invention is used for increasing and/or maintaining tissue homeostasis in the scalp, advantageously in the hair follicles, thus increasing the anchoring of the hair strands in the scalp. Advantageously, the extract of C. monspeliensis maintains and/or increases the cell differentiation in the scalp, more advantageously in the hair follicles, while maintaining and/or increasing the gene and/or protein expression of ECM1 in the hair follicles.

Thus, in a particularly advantageous embodiment, the extract of C. monspeliensis may be used for reducing hair loss.

The extract according to the invention may be all or part of the plant C. monspeliensis chosen from the whole plant, the aerial parts, the stalk, the leaves, the trichomes, the branches, the flowers and the seeds. Advantageously, it is an extract of the aerial parts. The term “aerial parts” means herein the leaves, the stalks, the branches and the flowers. More advantageously, the extract of C. monspeliensis is an extract of leaves.

The extract may be obtained using any extraction method known to those skilled in the art, chosen from hot decoction, milling including ultrasonic milling, using a mixer, maceration, extraction into water under subcritical conditions or extraction using a solvent. Advantageously, the extract will be obtained by maceration.

Advantageously, the extraction will not be performed by co-extraction with supercritical CO₂ in the presence of a solvent, in particular a solvent consisting of triglycerides of plant origin, more particularly a solvent consisting of C8-C10 triglycerides.

More advantageously, the extraction will not be performed using supercritical CO₂ or microwave hydrodistillation, in particular vacuum microwave hydrodistillation.

The term extraction under “subcritical conditions” means extraction in the presence of water, under temperature conditions of greater than 100° C. and pressure conditions of less than or equal to 22.1 MPa (221 bar), such that the water remains in the liquid state but has a viscosity and a surface tension lower than that of water at room temperature, increasing its dielectric constant.

Thus, the extraction pressure will be between 10 MPa (100 bar) and 25 MPa (250 bar).

The extraction may be performed using dry or fresh plant matter, advantageously dry plant matter, in an amount of from 0.1% to 30% by weight, advantageously from 1% to 20%, very advantageously from 5% to 15%, even more advantageously of 10% by weight of the plant matter relative to the total weight of the plant matter and of the extraction solvent.

The extraction may be performed at a temperature ranging from 4° C. to 300° C., including room temperature, that is to say a temperature of 20° C. In a preferential embodiment of the invention, the extraction will be performed at a temperature of from 60° C. to 90° C., preferentially from 70° C. to 85° C., more preferentially at a temperature of 80° C.

The extraction may be performed for a period of from a few seconds to 24 hours, preferentially from 1 minute to 12 hours, more preferentially for a period of from 5 minutes to 5 hours, and more advantageously for a period of from 15 minutes to 2 hours.

The solvent may be chosen from water, or a solvent mixture, preferably a polar protic solvent, and advantageously in water, an alcohol, a glycol, a polyol, a water/alcohol, water/glycol or water/polyol mixture (such as water mixed with ethanol, glycerol and/or butylene glycol and/or other glycols such as xylitol and/or propanediol, etc.), from 99/1 to 1/99 (w/w), advantageously in water as sole solvent.

In particular, the extract is obtained by aqueous extraction. For the purposes of the present invention, the term “extract obtained by aqueous extraction” means any extract obtained by extraction with an aqueous solution containing more than 60% by weight, advantageously at least 70% by weight, in particular at least 80% by weight, more particularly at least 90% by weight, particularly at least 95% by weight, of water relative to the total weight of the aqueous solution, even more advantageously not containing any glycol and/or polyol and in particular not containing any alcohol, more particularly containing only water.

In particular, the extract of Cistus monspeliensis according to the invention is an aqueous extract obtained in water or in a mixture of water and a solvent chosen from the group consisting of an alcohol, a glycol, a polyol and a mixture thereof, advantageously in water as the sole solvent. Advantageously, the extraction is not performed using boiling water.

In a particularly advantageous manner, the extract of Cistus monspeliensis according to the invention is a water-soluble extract. Thus, it is not a lipid extract. In particular, it does not contain essential oils or lipid fractions. Even more advantageously, the extract is not solidified as a wax, in particular it is not mixed with an oil of plant origin, more particularly consisting of C16-C18 triglycerides.

In one alternative embodiment, the extract is obtained by extraction in a mixture of propanediol and water in the respective proportion (80:20; v/v). In yet another alternative embodiment, the extract is obtained by extraction in a mixture of ethanol and water in the respective proportion (80:20; v/v).

In another alternative embodiment of the invention, the extraction may be performed in the presence of a nonionic surfactant, preferentially chosen from lauryl glucoside sold under the name Plantacare® 1200UP by BASF or caprylyl/capryl glucoside (Plantacare® 810 UP), preferentially caprylyl/capryl glucoside (Plantacare® 810 UP). The weight concentration of the nonionic surfactant may be between 0.5% and 5%, advantageously between 0.5 and 1%, more advantageously it will be 1% by weight relative to the total weight of the extract.

In yet another alternative embodiment of the invention, the extraction will be performed in water under subcritical conditions, at a temperature ranging from 100° C. to 300° C., advantageously from 120° C. to 250° C. The extraction may be performed at a single given temperature or at successive increasing temperatures. In an advantageous embodiment of the invention, the extraction will be performed at a single temperature of 120° C. In an alternative embodiment, it will be performed according to a gradient of three increasing temperatures of between 100° C. and 200° C., such as 120° C., 140° C. then 160° C., or 110° C., 130° C. then 150° C., or else 120° C., 145° C. then 170° C.

Very advantageously, the extract of C. monspeliensis may be obtained by maceration in water as the sole solvent.

Thus, in a first advantageous embodiment of the invention, the extract will be obtained from an amount of 10% by weight of the dried aerial parts of C. monspeliensis relative to the total weight of the plant and of the solvent, macerated in water as sole solvent, at a temperature of 80° C. for a period of 1 hour. The extract obtained is separated out by centrifugation and the supernatant is filtered (0.20 μm), under the conditions described in Example 1a).

In a second advantageous embodiment of the invention, the extract will be obtained from an amount of 20% by weight of the dried aerial parts of C. monspeliensis relative to the total weight of the plant and of the solvent, macerated in water as sole solvent, at a temperature of 80° C. for a period of 1 hour. The extract obtained is separated out by centrifugation and the supernatant is filtered (0.20 μm), under the conditions described in Example 1b).

In a third embodiment, the extract will be obtained from an amount of 10% by weight of the dried aerial parts of C. monspeliensis relative to the total weight of the plant and of the solvent, macerated in a water/propanediol mixture (20:80; v/v), at a temperature of 4° C. for a period of 2 hours. The extract obtained is separated out by centrifugation and the supernatant is filtered (0.20 μm), under the conditions described in Example 1c).

In a fourth embodiment of the invention, the extract will be obtained from an amount of 10% by weight of the dried leaves of C. monspeliensis relative to the total weight of the plant and of the solvent, macerated in water as solvent, at a temperature of 4° C. for a period of 24 hours. The extract obtained is separated out by centrifugation and the supernatant is filtered (0.20 μm), under the conditions described in Example 1d).

In a fifth embodiment of the invention, the extract will be obtained from an amount of 10% by weight of the dried aerial parts of C. monspeliensis relative to the total weight of the plant and of the solvent, extracted into water under subcritical conditions at a temperature of 120° C. under a pressure of 250 bar (25 MPa). The crude extract is decanted, centrifuged and then filtered (0.20 μm), under the conditions of Example 1e).

In an advantageous embodiment of the invention, the extract obtained is dried and sprayed in the presence of maltodextrin. Maltodextrin is present in a concentration of between 70% and 90%, advantageously between 70% and 80%, more advantageously between 75% and 80%; very advantageously, the maltodextrin concentration is 80% by weight relative to the total weight of maltodextrin and of the extract according to the invention. The extract obtained after spraying is in powder form.

In a very advantageous embodiment of the invention, the extract of C. monspeliensis according to the invention does not contain any labdane (C₂₀H₃₈; molar mass 278.524 g/mol; CAS No. 561-90-0) or any labdanic and/or labdenoic acid derivatives thereof and/or salts thereof, in particular the alkali metal salts such as the sodium and potassium salts, the alkaline-earth metal salts such as the calcium salts and the magnesium salts, the ammonium salts, the monomethylammonium, dimethylammonium, trimethylammonium and dicyclohexylammonium salts.

In particular, the extract of C. monspeliensis according to the invention does not contain any labdanic and/or labdenoic acid derivatives chosen from:
labdanolic acid also known as labdanic acid (C₂₀H₃₆O₃; molar mass 324.505 g/mol; CAS No. 10267-24-0) or a salt thereof;
labdane-8α,15-diol (C₂₀H₃₈O₂; molar mass 310.522 g/mol; CAS No. 10267-22-8) or a salt thereof;
labd-15-enoic acid (C₂₀H₃₆O₂; molar mass 308.27 g/mol; CAS No. 24460-80-8) or a salt thereof;
1-naphthalenepentanoic acid, 3,4,4a,5,6,7,8,8a-octahydro-β,2,5,5,8a-pentamethyl, (4aS,8aS), also known as labd-8-en-15-oic acid (CAS No. 248581-33-1, molar mass 306.26 g/mol, C₂₀H₃₄O₂) or a salt thereof;
1-naphthalenepentanoic acid, 3,4,4a,5,6,7,8,8a-octahydro-β,2,5,5,8a-pentamethyl, methyl ester, (4aS,8aS) (CAS No. 248581-34-2, C₂₁H₃₆O₂, molar mass: 320.27 g/mol) or a salt thereof;
1-naphthalenepentanoic acid, 3,4,4a,5,6,7,8,8a-octahydro-β,2,5,5,8a-pentamethyl, ethyl ester, (4aS,8aS) (CAS No. 248581-35-3, C₂₂H₃₈O₂, molar mass: 334.29 g/mol) or a salt thereof;
1-naphthalenepentanoic acid, 1,4,4a,5,6,7,8,8a-octahydro-β,2,5,5,8a-pentamethyl, (1S,4aS,8aS) also known as labd-7-en-15-oic acid (CAS No. 248581-36-4, C₂₀H₃₄O₂, molar mass: 306.26 g/mol) or a salt thereof;
1-naphthalenepentanoic acid, 1,4,4a,5,6,7,8,8a-octahydro-β,2,5,5,8a-pentamethyl, methyl ester, (1S,4aS,8aS)— (CAS No. 248581-37-5), C₂₁H₃₆O₂, molar mass: 320.27 g/mol) or a salt thereof;
1-naphthalenepentanoic acid, 1,4,4a,5,6,7,8,8a-octahydro-β,2,5,5,8a-pentamethyl-, ethyl ester, (1S,4aS,8aS) (CAS No. 248581-38-6, C₂₂H₃₈O₂, molar mass: 334.29 g/mol) or a salt thereof;
1-naphthalenepentanoic acid, decahydro-β,5,5,8a-tetramethyl-2-methylene-, ethyl ester, (1S,4aS,8aS) (CAS No. 248581-39-7, C₂₂H₃₈O₂, molar mass: 334.29 g/mol) or a salt thereof;
1-naphthalenepentanoic acid, decahydro-β2,5,5,8a-pentamethyl, ethyl ester, (1S,2S,4aS,8aR) (CAS No. 248581-40-0, C₂₂H₄₀O₂, molar mass: 336.30 g/mol) or a salt thereof;
1-naphthalenepentanoic acid, decahydro-β5,5,8a-tetramethyl-2-methylene-, (1S,4aS,8aS) (CAS No. 248606-63-5), C₂₀H₃₄O₂, molar mass: 306.26 g/mol) or a salt is thereof or the isomer thereof eperuic acid also known as 1-naphthalenepentanoic acid, decahydro-β,5,5,8a-tetramethyl-2-methylene-, β,1R,4aR,8aR) or a salt thereof (C₂₀H₃₄O₂; molar mass 306.49 g/mol; CAS No. 20784-69-4).

In particular, it does not comprise any labdanic derivatives of general formula I below:

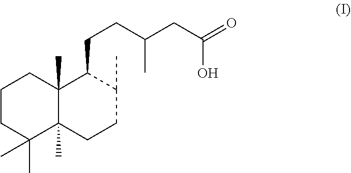

in which two of the three dashed lines correspond to a single bond, the third dashed line corresponding to a double bond, and the salts of these derivatives.

In particular, it does not comprise either any labdanic derivatives of general formula 1 below or the salts of these derivatives:

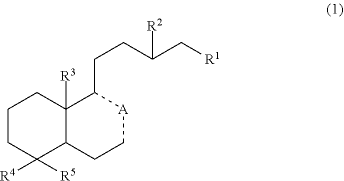

in which R¹ represents a group —CH₂OH, —COOR⁶ or —COOX, X representing the base which may form a salt, R⁶ represents a hydrogen atom or an alkyl group containing from 1 to 3 carbon atoms, R² to R⁵ representing, independently of each other, a hydrogen atom or a methyl group and -A- represents a ═C(CH₃)—, —C(CH₃)═, —C(═CH₂)—, —CH(CH₃)—, —C(OH) or (CH₃)— group.

In particular, the extract of C. monspeliensis according to the invention does not contain any β-endorphin.

More advantageously, the extract of C. monspeliensis obtained in water as sole solvent, more advantageously the extract of the aerial parts prepared under the io conditions of Example 1a), does not contain any labdane or any labdanic derivatives and/or salts thereof, advantageously chosen from those mentioned above.

More advantageously, the extract of C. monspeliensis obtained in water as sole solvent, more advantageously the extract of the aerial parts prepared under the conditions of Example 1a), does not contain any β-endorphin.

In particular, the cosmetic composition comprising the extract of C. monspeliensis, advantageously obtained in water as sole solvent, more advantageously the extract of the aerial parts prepared under the conditions of Example 1a), does not contain any labdane or any labdanic derivatives and/or salts thereof, advantageously chosen from those mentioned above.

In particular, the cosmetic composition comprising the extract of C. monspeliensis, advantageously obtained in water as sole solvent, more advantageously the extract of the aerial parts prepared under the conditions of Example 1a), does not contain any β-endorphin.

The extract according to the invention may be used alone or included in a cosmetic composition. If the extract is used alone as a cosmetic active ingredient, it is advantageously dissolved in an aqueous solution comprising glycerol, advantageously present at a concentration of from 60% to 90%, more advantageously from 70% to 85%, very advantageously at a concentration of 80% by weight relative to the total weight of the water-glycerol solution comprising the extract.

In an advantageous embodiment, the extract of C. monspeliensis according to the invention is not used in combination with Thymus hyemalis or an extract thereof. Even more advantageously, the cosmetic composition according to the invention does not contain any Thymus hyemalis or an extract thereof.

In an alternative embodiment of the invention, the extract will be dissolved and/or diluted in a solvent, notably a polar solvent, such as water, an alcohol, a polyol, a glycol, such as pentylene glycol and/or butylene glycol and/or hexylene glycol and/or caprylyl glycol, or a mixture thereof, preferentially a water-glycol mixture, more preferentially containing a glycol chosen from hexylene glycol, caprylyl glycol and mixtures thereof. Advantageously, the extract obtained is diluted and/or soluble in an aqueous solution containing hexylene glycol, in particular containing between 0.1% and 10% by weight of hexylene glycol, preferentially between 0.5% and 5% by weight of hexylene glycol, relative to the total weight of the cosmetic ingredient. Advantageously, the extract obtained is diluted and/or soluble in an aqueous solution containing caprylyl glycol, in particular containing between 0.01% and 5% by weight of caprylyl glycol, preferentially between 0.1% and 1% by weight of caprylyl glycol, relative to the total weight of the aqueous solution comprising the extract.

Alternatively, the extract of C. monspeliensis may be included in a cosmetic composition comprising at least one cosmetically acceptable excipient. The term “cosmetically acceptable excipient” means a cosmetic excipient that is non-irritant to the skin, which does not induce an allergic response and which is chemically stable. The excipient(s) may be chosen from surfactants and/or emulsifiers, preserving agents, buffers, chelating agents, denaturing agents, opacifiers, pH modifiers, reducing agents, stabilizers, thickeners, gelling agents, film-forming polymers, fillers, mattifying agents, gloss agents, pigments, colorants, fragrances, and mixtures thereof. The CTFA (Cosmetic Ingredient Handbook, Second Edition (1992)) describes various cosmetic excipients that are suitable for use in the present invention.

Advantageously, the excipient(s) are chosen from the group comprising polyglycerols, esters, cellulose polymers and derivatives, lanolin derivatives, phospholipids, lactoferrins, lactoperoxidases, sucrose-based stabilizers, vitamin E and derivatives thereof, xanthan gum, natural and synthetic waxes, plant oils, triglycerides, unsaponifiable matter, phytosterols, silicones, protein hydrolysates, betaines, amine oxides, plant extracts, sucrose esters, titanium dioxides, glycines, and parabens, and more preferably from the group consisting of steareth-2, steareth-21, glycol-15 stearyl ether, cetearyl alcohol, phenoxyethanol, methylparaben, ethylparaben, propylparaben, butylparaben, butylene glycol, caprylyl glycol, natural tocopherols, glycerol, dihydroxycetyl sodium phosphate, isopropyl hydroxycetyl ether, glycol stearate, triisononanoin, octyl cocoate, polyacrylamide, isoparaffin, laureth-7, a carbomer, propylene glycol, hexylene glycol, glycerol, bisabolol, a dimethicone, sodium hydroxide, PEG-30 dipolyhydroxystearate, caprylic/capric triglycerides, cetearyl octanoate, dibutyl adipate, grape seed oil, jojoba oil, magnesium sulfate, EDTA, a cyclomethicone, xanthan gum, citric acid, sodium lauryl sulfate, mineral waxes and oils, isostearyl isostearate, propylene glycol dipelargonate, propylene glycol isostearate, PEG 8, beeswax, glycerides from hydrogenated palm kernel oil, lanolin oil, sesame oil, cetyl lactate, lanolin alcohol, castor oil, titanium dioxide, lactose, sucrose, low density polyethylene, an isotonic saline solution, and mixtures thereof.

The cosmetic composition may be chosen from an aqueous or oily solution, a cream or an aqueous gel or an oily gel, notably a shower gel, a milk, an emulsion, a microemulsion or a nanoemulsion, which is notably oil-in-water or water-in-oil or multiple or silicone-based, a mask, a serum, a lotion, a liquid soap, a shampoo, an ointment, a foam, a patch, an anhydrous product, which is preferably liquid, pasty or solid, for example in the form of makeup powders, a wand or a stick, notably in the form of a lipstick. Advantageously, it will be a cream or a serum.

The cosmetic composition may also comprise active ingredients for reinforcing the barrier function of the skin and/or the skin appendages, inducing a complementary or synergistic effect with the extract of C. monspeliensis according to the invention, chosen from those which reinforce the barrier function and decrease the transepidermal water losses and/or those which increase the water content of the skin and/or of the mucous membranes and/or stimulate aquaporin synthesis in order to improve the circulation of water in the cells. Mention will be made of serine, urea and its derivatives, products such as microspheres of marine collagen and of chondroitin sulfate as a glycosaminoglycan, a formulation comprising a hyaluronic acid salt, urea, trehalose, glyceryl triacetate and Polyquaternium-51 sold, respectively, under the names Marine Filling SpheresTM and Advanced Moisturizing Complex™; hyaluronic acid microspheres sold under the name Hyaluronic Filling SpheresTM; an extract of red alga or a mixture of acacia polysaccharide, alginate and serine sold, respectively, under the names Osmogelline™ and Micropatch™ Serine; or a combination of pullulan, of hyaluronic acid or a salt or derivative thereof and of alginic acid, or a salt or derivative thereof, sold under the name PatcH20™. Other compounds may be used, such as alkylcelluloses, lecithins, sphingoid-based compounds, ceramides, phospholipids, cholesterol and its derivatives, glycosphingolipids, phytosterols (stigmasterol and β-sitosterol, campesterol), essential fatty acids, 1,2-diacylglycerol, 4-chromanone, pentacyclic triterpenes, such as ursolic acid, petroleum jelly, lanolin, sugars, in particular trehalose and its derivatives, rhamnose, fructose, maltose, lactose, erythritol, mannitol, D-xylose and glucose, adenosine and its derivatives, sorbitol, polyhydric alcohols, which are advantageously C₂-C₆, and more advantageously C₃-C₆, such as glycerol, propylene glycol, 1,3-butylene glycol, dipropylene glycol, diglycerol, polyglycerol and a mixture thereof, glycerol and its derivatives, glyceryl polyacrylate, sodium lactate, pentanediol, serine, lactic acids, AHAs, BHAs, sodium pidolate, xylitol, sodium lactate, ectoin and its derivatives, chitosan and its derivatives, collagen, plankton, steroid derivatives (including DHEA, its 7-oxidized and/or 17-alkylated derivatives and sapogenins), methyl dihydrojasmonate, vitamin D and its derivatives, an extract of Malva sylvestris or an extract of Centella asiatica, acrylic acid homopolymers, 3-glucan and in particular sodium carboxymethyl β-glucan, a C-glycoside derivative such as those described in patent application WO 2002/051828, a musk rose oil, an extract of the microalga Prophyridium cruentum enriched with zinc, sold by Vincience under the name Algualane Zinc™, arginine, acetyl hexapeptide sold by Lipotech under the name Diffuporine™, the hydrolysate of Viola tricolor sold by Silab under the name Aquaphyline™, or a polysaccharide extracted from Cassia angustifolia seeds, sold under the name Hyalurosmooth™ by the Applicant, a fermented hydrolysate of Saccharomyces cerevisia esold under the name Relipidium™, or else one or more of the compounds of natural moisturizing factor or a natural extract of honey sold by the Applicant under the name Melhydran™.

Other types of active agents may be present in the composition, such as anti-ageing active agents and/or bleaching active agents and/or antipollution active agents and/or active agents which promote the radiance of the complexion.

These agents may be, for example, a leaf extract of Cassia alata sold under the name DN-Age™ and/or an extract of lychees sold under the name Litchiderm™ as antioxidant active agents, a combination of an extract of Salvia miltiorhizza and of niacinamide, sold under the name CollRepair™ as a deglycating agent, an anti-wrinkle extract of Cichorium intybus sold under the name Lox-Age™, an extract of Achillea millefolium sold under the name Neurobiox™, an extract of Polygonum bistotta sold under the name Perlaura™, an extract of Alpinia galanga sold under the name Hyalufix™, an extract of corn sold under the name Deliner™ or an extract of Voandzeia subterranea sold under the name Epigenist™ by the Applicant or else active agents which promote the firmness of the skin, such as a synthetic tetrapeptide sold under the name Dermican™, an extract of Hibiscus abelmoschus sold under the name Linefactor™, a purified extract of pea sold under the name Proteasyl™, an extract of Manilkara multinervis sold under the name Elestan™ a pulp extract of Argania spinosa sold under the name Argassential™ by the Applicant. An extract of the plant Origanum majorana sold under the name Dermagenist™ and/or an extract of Khaya senegalensis sold under the name Collalift®18 may also be added to the cosmetic composition.

In a preferential embodiment of the invention, the extract of C. monspeliensis will be present in the cosmetic composition in a concentration of from 0.0001% to 20% by weight, preferentially from 0.001% to 5% by weight and more preferentially from 0.01% to 3% by weight relative to the total weight of the cosmetic composition.

The cosmetic composition comprising the extract of C. monspeliensis according to the invention may thus be used for increasing and/or maintaining tissue homeostasis in the skin and/or the mucous membranes and/or the skin appendages. Said composition is thus used for maintaining and/or increasing the gene and/or protein expression of ECM1 and/or of involucrin in order to increase and/or maintain the barrier function of the skin and/or of the skin appendages and/or cell differentiation in the skin and/or the mucous membranes and/or the skin appendages. It may also be used for increasing and/or maintaining tissue renewal in the skin and/or the mucous membranes and/or the skin appendages and/or for improving the surface appearance of the skin and/or of the mucous membranes and/or of the skin appendages and/or for reducing hair loss, and/or for increasing the radiance and/or homogeneity of the skin complexion and/or for reducing water losses in the skin and/or the skin appendages and/or for preventing dehydration therefrom, and/or for reducing pigmentation marks.

In an advantageous embodiment, said cosmetic composition is not for increasing and/or maintaining cell growth or proliferation, or cell division. In a particular embodiment, the cosmetic composition is not for increasing and/or maintaining the synthesis of ATP.

In another advantageous embodiment, said cosmetic composition does not comprise any alginate, or algin, or hydrolysed algin, or any extract of a plant chosen from Lavandula stoechas, Helichrysum italicum and mixtures thereof.

A second subject of the invention relates to a non-therapeutic cosmetic care process comprising the topical or oral administration, preferentially the topical application, of an extract of C. monspeliensis or of a cosmetic composition comprising same for increasing and/or maintaining tissue homeostasis in the skin and/or the mucous membranes and/or the skin appendages.

The cosmetic care process advantageously makes it possible to increase and/or maintain the barrier function of the skin and/or of the skin appendages and/or to increase cell differentiation in the skin and/or the mucous membranes and/or the skin appendages, by maintaining and/or increasing the gene and/or protein expression of involucrin and/or of ECM1.

It also advantageously makes it possible to increase and/or maintain tissue renewal in the skin and/or the mucous membranes and/or the skin appendages.

It also advantageously makes it possible to improve the surface appearance of the skin and/or of the mucous membranes and/or of the skin appendages and/or to reduce hair loss, and/or to increase the radiance and/or homogeneity of the skin complexion and/or to reduce water losses in the skin and/or the skin appendages and/or to prevent dehydration therefrom, and/or to reduce pigmentation marks.

In one embodiment of the invention, the cosmetic care process comprises the topical application of the extract of C. monspeliensis or of the cosmetic composition comprising same to all or part of the face and/or the body and/or the scalp and/or the skin appendages and/or the mucous membranes, chosen from the legs, the hands, the thighs, the stomach, the neckline, the neck, the arms, the torso, the back, the hair, the face, notably the area around the eyes, advantageously the neckline and/or the face and notably the area around the eyes, even more advantageously the area around the eyes.

Advantageously, a subject of the invention is also a cosmetic treatment method for increasing and/or maintaining tissue homeostasis in the skin and/or the mucous membranes and/or the skin appendages of an individual in need thereof/who so desires, in particular for increasing and/or maintaining the gene and/or protein expression of involucrin and/or of ECM1, for increasing and/or maintaining the barrier function of the skin and/or the skin appendages and/or cell differentiation in the skin and/or the mucous membranes and/or the skin appendages, of an individual in need thereof/who so desires, more particularly for increasing and/or maintaining tissue renewal in the skin and/or the mucous membranes and/or the skin appendages, of an io individual in need thereof/who so desires, even more particularly for improving the surface appearance of the skin and/or for reducing hair loss and/or for increasing the radiance of the skin complexion and/or for reducing water losses in the skin and/or the skin appendages and/or for preventing dehydration thereof, in an individual in need thereof/who so desires, comprising the steps of:
identifying on the individual an area of skin and/or of mucous membranes and/or of skin appendages for which it is desired to increase and/or maintain the tissue homeostasis, and/or to increase and/or maintain the gene and/or protein expression of involucrin and/or of ECM1, and/or to increase and/or maintain the barrier function and/or cell differentiation, and/or to increase and/or maintain tissue renewal and/or to improve the surface appearance and/or to reduce hair loss, and/or to increase the radiance of the skin complexion and/or to reduce the water losses and/or to prevent dehydration thereof, and
applying topically to this area of skin and/or of mucous membranes and/or of skin appendages a cosmetic composition containing the extract of Cistus monspeliensis according to the invention in an amount that is effective for increasing and/or maintaining tissue homeostasis, and/or increasing and/or maintaining the gene and/or protein expression of involucrin and/or of ECM1, and/or increasing and/or maintaining the barrier function and/or cell differentiation, and/or increasing and/or maintaining tissue renewal and/or improving the surface appearance and/or reducing hair loss and/or increasing the radiance of the complexion and/or reducing the water losses and/or preventing the dehydration of this area of skin and/or of mucous membranes and/or of skin appendages.

A third subject of the invention relates to the extract of C. monspeliensis according to the invention or a dermatological or pharmaceutical composition comprising same, for its use for preventing and/or treating diseases induced by a decrease in the gene and/or protein expression of ECM1, advantageously lichen sclerosus or lipoid proteinosis, and/or for preventing and/or treating scars.

In one embodiment, the extract is included in the dermatological or pharmaceutical composition in a concentration of from 0.0001% to 20% by weight, preferentially from 0.001% to 5% by weight and more preferentially from 0.01% to 3% by weight relative to the total weight of the composition.

The dermatological composition or the pharmaceutical composition will comprise at least one dermatologically or pharmaceutically acceptable excipient.

In one embodiment of the invention, the extract of C. monspeliensis or the dermatological or pharmaceutical composition comprising same is applied topically to all or part of the face and/or the body and/or the scalp and/or the skin appendages and/or the mucous membranes, chosen from the legs, the hands, the thighs, the stomach, the neckline, the neck, the arms, the torso, the back, the hair, the face, notably the area around the eyes, advantageously the neckline and/or the face and notably the area around the eyes, more advantageously the area around the eyes.

In an advantageous embodiment of the invention, the extract of C. monspeliensis is an extract of the aerial parts of C. monspeliensis, more advantageously an extract of leaves. More advantageously, the extract is an aqueous extract obtained in water as sole solvent.

Examples referring to the description of the invention are presented below. These examples are given for illustrative purposes and shall in no way limit the scope of the invention. Each example has a general scope. The examples form an integral part of the present invention, and any feature appearing to be novel relative to any prior art, from the description taken in its entirety, including the examples, forms an integral part of the invention.

Unless otherwise indicated, the percentages are expressed on a weight/weight basis.

### EXAMPLES

### Example 1: Preparation of Extracts of C. Monspeliensis

The plant C. monspeliensis originates from Morocco.

Example 1a) 10% by weight of dried aerial parts of the plant C. monspeliensis, relative to the total weight of the plant parts and of the solvent, were macerated in water as sole solvent at a temperature of 80° C., for a period of 1 hour. The extract obtained was separated out by centrifugation and the supernatant was filtered (0.20 μm). The extract is in liquid form.

Example 16b) 20% by weight of dried aerial parts of the plant C. monspeliensis, relative to the total weight of the plant parts and of the solvent, were macerated in water as sole solvent at a temperature of 80° C., for a period of 1 hour. The extract obtained was separated out by centrifugation and the supernatant was filtered (0.20 μm). The extract is in liquid form.

Example 1c) 10% by weight of dried aerial parts of the plant C. monspeliensis, relative to the total weight of the plant parts and of the solvent, were macerated in a water/propanediol mixture (20:80; v/v) as sole solvent at a temperature of 4° C., for a period of 2 hours. The extract obtained was separated out by centrifugation and the supernatant was filtered (0.20 μm). The extract is in liquid form.

Example 1d) 10% by weight of dried leaves, relative to the total weight of the plant part and of the solvent, were macerated in water as sole solvent at a temperature of 4° C., for a period of 24 hours. The extract obtained was separated out by centrifugation and the supernatant was filtered (0.20 μm). The extract is in liquid form.

Example 1e) 10% by weight of dried aerial parts of the plant C. monspeliensis, relative to the total weight of the plant parts and of the solvent, were extracted into water under subcritical conditions, at a temperature of 120° C. under a pressure of 250 bar. The crude extract is decanted, centrifuged and then filtered. The extract is in liquid form.

Each of these extracts described above may then be dried and sprayed in the presence of maltodextrin (from 70% to 90% by weight, advantageously 80% by weight, relative to the total weight of maltodextrin and of the extract). The extract obtained after spraying will be in powder form.

### Example 2: Increase in the Gene and Protein Expression of the Marker ECM1 in the Presence of an Extract of C. Monspeliensis

### Example 2a) Increase in the Gene Expression of ECM1

Protocol. Human keratinocytes obtained from healthy female donors (from 39 to 60 years old) were cultivated until confluence was reached (5-10 days of culturing) in a defined growth medium (Dulbecco's Modified Eagle Medium: DMEM) supplemented with 10% foetal calf serum and antibiotics, in a controlled environment (37° C., 5% CO₂, relative humidity>95%). The cells were then treated with the extract of C. monspeliensis prepared under the conditions described in Example 1a) at a final concentration in the medium of 0.001% by weight relative to the final volume of the medium, for a period of 48 hours, or were not treated with the extract (untreated control). The cells were recovered and frozen at −80° C. until the time of use.

The total RNA was extracted using the NucleoSpin 96 RNA kit (Machery-Nagel GmbH & Co KG). The quantification and quality of the total RNA were controlled by measuring the optical densities at 260 and 280 nm.

The expression of the ECM1 gene was quantified by qRT-PCR. The messenger mRNAs were transcribed into cDNA with specific oligonucleotides (SYBR Green) using a thermocycler (LightCycler®480 I Master system (Roche Molecular Diagnostics). The genes ACTB (Actin B) and EEF1A1 (Eukaryotic Translation Elongation Factor 1 Alpha 1) were used as reference genes for normalization of the results. The fluorescence was measured on each amplification cycle. The relative quantification was performed by means of the ΔΔCt method after producing the calibration curve.

The results are expressed as the mean±standard deviation of the mean on five different keratinocyte cultures and are indicated in Table 1 below. The statistical study of the differences was performed using Student's t test versus the untreated control (control (without extract)).

TABLE 1

| | MEAN | SD |
|---|---|---|
| Control (with out extract) | 100 | 0 |
| Extract of C. monspeliensis prepared according to Ex. 1a) | 152* | 9 |
| (1 × 10⁻³% w/v) | | |

*P < 0.001

Conclusion: The extract of C. monspeliensis increased the gene expression of ECM1 by at least 43% relative to the control, showing its capacity for increasing cell differentiation, thus participating in tissue homeostasis.

### Example 2b) Increase in the Protein Expression of ECM1

Protocol. Human keratinocytes obtained from a healthy female donor (from 39 to 60 years old) were cultivated in a defined medium (DMEM) until confluence was reached (4 days of culturing) and then placed or not placed in contact with the extract of C. monspeliensis prepared according to Example 1a) (final concentration of 1×10⁻³% by weight relative to the final volume of the medium) for a period of 48 hours. The same culture medium without the addition of extract was used as a control (untreated control).

The cells were subsequently harvested and then lysed with a specific lysis buffer in order to perform the immunolocalization (Western blotting). The total protein concentration was determined by means of the BCA method, and each condition is deposited at the same concentration. The proteins were identified by capillary electrophoresis (ProteinSimple, USA) using an anti-ECM1 primary antibody and immunolocalized using a peroxidase-coupled conjugated secondary antibody. The results were quantified using the Compass Software (version 2.7.1 (ProteinSimple)), and reported relative to the untreated control (control (without extract)). The results are expressed as the mean±standard deviation of the mean on two different keratinocyte cultures (n=4) and are indicated in Table 2 below. The statistical study was performed using the Sigmaplot™ software.

TABLE 2

| | MEAN | SD |
|---|---|---|
| Control (without extract) | 100 | 2 |
| Extract of C. monspeliensis prepared according to Ex. 1a) | 139* | 6 |
| (1 × 10⁻³% w/v) | | |

*p < 0.001

Conclusion: The extract of C. monspeliensis increased the expression of ECM1 in the keratinocytes in culture by at least 31%, confirming the capacity of the extract for increasing cell differentiation and consequently tissue homeostasis.

### Example 3: Increasing the Protein Synthesis of Involucrin in the Presence of an Extract of C. Monsneliensis

Protocol. Human keratinocytes obtained from healthy adult female donors (45 and 46 years old) were cultivated in a defined growth medium (MCDB153 standard medium) supplemented with 2% foetal calf serum in a controlled environment (37° C., 5% CO₂, relative humidity>95%) for a period of 3 to 4 days. The culture medium was replaced with serum-free medium and treated or not treated with the extract of C. monspeliensis prepared according to Example 1a) (final concentration of 1×10⁻³% by weight relative to the final volume of the medium) for a period of 3 days.

The level of production of involucrin was quantified by means of the ELISA method on cell homogenates.

The results are expressed as the mean percentage±standard deviation relative to the untreated control (control (without extract)) (n=3) and are indicated in Table 3 io below. The statistical study was performed using the Sigmaplot™ software.

TABLE 3

| | MEAN | SD |
|---|---|---|
| Control (without extract) | 100 | 15 |
| Extract of C. monspeliensis prepared according to Ex. 1a) | 390* | 107 |
| (1 × 10⁻³% w/v) | | |

*p < 0.01

Conclusion: The extract of C. monspeliensis increased the synthesis of involucrin by at least 168% in the cultivated keratinocytes. The extract of C. monspeliensis according to the invention is effective for increasing the barrier function of the skin and/or of the skin appendages.

### Example 4: In Vivo Evaluation of Tissue Renewal in the Presence of an Extract of C. Monspeliensis

Protocol. The effect of a clinical treatment with a formulation comprising the extract of C. monspeliensis on tissue renewal was studied by using the DHA (dihydroxyacetone) method. The principle is to colour the skin surface (epidermis) by using this molecule used as a self-tanning molecule. The chemical reaction between DHA and the amino acids of the proteins of the epidermis forms brown-coloured melanoidin pigments. The colour was evaluated using a chromameter (Chromameter CR-400). The decolorization time was used to estimate the time required for the epidermis to become renewed.

Women from 45 to 65 years old of light phototype (Type I and II according to Fitzpatrick) were included in the analysis. The volunteers applied to each forearm, twice a day for a period of 28 days, a formulation comprising the extract of C. monspeliensis prepared under the conditions of Example 1a) (0.1% by weight relative to the total weight of the formulation of Example 5a) or of the same formulation not containing the extract according to the invention (control).

The parameter ΔE was calculated to measure the colour difference between a measurement at time x (Dx) and the measurement at time 0 (DO: basal reference level). The time required to reach a value ΔE=0 corresponds to the renewal time of the epidermis. The angle ITA was calculated to measure the difference in brown colour before and after application of the product.

The results correspond to the number of days required for total renewal of the epidermis (ΔE=0), after 28 days of application of the formulation comprising the extract of C. monspeliensis or without the extract (control), and are indicated in Table 4 below.

TABLE 4

| | MEAN |
|---|---|
| Control | 20.3 |
| Extract of C. monspeliensis Example 1a) 0.1% (w/w) | 16.9* |

*p < 0.1

Conclusion: After 28 days of application of the formulation comprising the extract of C. monspeliensis, the renewal time of the epidermis was reduced by 3.4 days in comparison with the control. The extract is thus effective for increasing tissue renewal.

### Example 5: Examples of Cosmetic or Dermatological Compositions Comprising an Extract of Cistus Monspeliensis

### Example 5a) Cosmetic Formulation

The percentages are expressed in Table 5 on a weight basis relative to the total weight of the cosmetic formulation.

TABLE 5

| Water | QS 100 |
|---|---|
| Plant oil | 4.00 |
| Cocoyl caprylate/caprate | 4.00 |
| Dicaprylyl ether | 4.00 |
| Propylene glycol, phenoxyethanol, chlorphenesin, methylparaben | 2.50 |
| Sodium polyacrylate | 1.00 |
| Hydrogenated plant glycerides | 1.00 |
| Sodium stearoyl glutamate | 0.05 |
| Extract of C. monspeliensis Ex. 1a) | 0.10 |

### Example 5b) Anti-Ageing Cream

The percentages are expressed in Table 6 on a weight basis relative to the total weight of the cream (% w/w).

TABLE 6

| Phase A | |
|---|---|
| Sucrose polystearate, cetyl palmitate | 2.50 |
| Sodium stearate glutamate | 0.50 |
| Cetearyl alcohol | 1.00 |
| Pentaerythrityl distearate | 1.5 |
| Dicaprylyl ether | 3.00 |
| Dibutyl adipate | 5.00 |
| Propylheptyl caprylate | 5.00 |
| Phenoxyethanol, ethylhexylglycerol | 1.00 |
| bis-Ethylhexyloxyphenol methoxyphenyl triazine | 1.50 |
| Phase B | |
| Water | 64.40 |
| Glycerol | 3.00 |
| Xanthan gum | 0.30 |
| Disodium EDTA | 0.20 |
| Phase C | |
| Methylenebis(benzotriazolyl)(tetramethylbutyl)phenol (nano), | 6.00 |
| water, decyl glucoside, propylene glycol, xanthan gum | |
| Phase D | |
| Undecane, tridecane | 2.00 |
| Fluorphlogopite, lauroyllysine | 1.00 |
| Phase E | |
| Water | 2.00 |
| Extract of C. monspeliensis Ex. 1a), maltodextrin | 0.10 |

The cream is prepared by the usual methods in the field well known to those skilled in the art, by mixing the five phases.

### Example 6: Increasing the Radiance and Homogeneity of the Complexion

Protocol. B16 melanocytes were cultivated in a standard culture medium comprising foetal calf serum for a period of 3 days at a temperature of 37° C. (5% CO₂). The medium was renewed identically with addition of the extract of C. monspeliensis according to Example 1a) at a final concentration of 0.01% by weight relative to the final volume of the medium or without any extract (control medium). After a period of 3 days of culturing, the total amount of melanin was measured by optical density at 475 nm. The tests were performed in triplicate and the results expressed as the percentage mean relative to the control (culture without any extract of C. monspeliensis) (SD: standard deviation). The statistical analysis was performed by Student's test (Sigmaplot™)

TABLE 7
Results:

| | MEAN | SD |
|---|---|---|
| Control | 100 | 0 |
| Extract of C. monspeliensis 0.01% w/v Ex. 1a) | 38* | 7 |

*p < 0.001

Conclusion: the extract reduced the total amount of melanin by at least 55% in the melanocytes analysed, demonstrating the effect of the extract on the homogeneity and radiance of the complexion.

1.-17. (canceled)

18. A cosmetic method for increasing and/or maintaining tissue homeostasis in the skin and/or the mucous membranes and/or the skin appendages comprising administering an effective amount of an extract of the aerial parts of Cistus monspeliensis or of a cosmetic composition containing it to an individual.

19. The cosmetic method according to claim 18, wherein the method increases and/or maintains the gene and/or protein expression of involucrin and/or of ECM1, increases and/or maintains the barrier function of the skin and/or of the skin appendages and/or promoting cell differentiation in the skin and/or the mucous membranes and/or the skin appendages.

20. The cosmetic method according to claim 18, wherein the extract increases and/or maintains tissue renewal in the skin and/or the mucous membranes and/or the skin appendages.

21. The cosmetic method according to claim 18, wherein the method improves the surface appearance of the skin and/or the mucous membranes and/or the skin appendages.

22. The cosmetic method according to claim 18, wherein the method reduces hair loss and/or increases the radiance of the skin complexion.

23. The cosmetic method according to claim 18, wherein the method reduces water losses in the skin and/or the skin appendages and/or prevents the dehydration thereof.

24. The cosmetic method according to claim 18, wherein the extract of Cistus monspeliensis is an extract of the leaves.

25. The cosmetic method according to claim 18, wherein the extract of Cistus monspeliensis is an aqueous extract obtained in water or a mixture of water and a solvent chosen from the group consisting of an alcohol, a glycol, a polyol and a mixture thereof.

26. The cosmetic method according to claim 25, wherein the extract of Cistus monspeliensis is an aqueous extract obtained in water as the sole solvent.

27. The cosmetic method according to claim 18, wherein the cosmetic composition also comprises at least one cosmetically acceptable excipient, the extract being present in a concentration of from 0.0001% to 20% by weight, relative to the total weight of the cosmetic composition.

28. The cosmetic method according to claim 27, wherein the extract is present in the cosmetic composition in a concentration of from 0.001% to 5% by weight, relative to the total weight of the cosmetic composition.

29. The cosmetic method according to claim 18, wherein the administration is a topical application.

30. The cosmetic method according to claim 18, wherein the administration is a topical application to all or part of the face and/or the body and/or the scalp and/or the skin appendages and/or the mucous membranes, chosen from the legs, the hands, the thighs, the stomach, the neckline, the neck, the arms, the torso, the back, the hair and the face.

31. The cosmetic method according to claim 18, wherein the administration is a topical application to the neckline and/or the face.

32. The cosmetic method according to claim 18, wherein the administration is a topical application to the area around the eyes.

33. A treatment method for preventing and/or treating diseases induced by a decrease in the gene and/or protein expression of ECM1, and/or for preventing and/or treating scars comprising administering an effective amount of an extract of the aerial parts of Cistus monspeliensis or of a dermatological or pharmaceutical composition containing it to an individual.

34. The method according to claim 33, wherein the extract of Cistus monspeliensis is present in a dermatological or pharmaceutical composition in a concentration of from 0.0001% to 20% by weight, relative to the total weight of the composition.

35. The method according to claim 33, wherein the extract of Cistus monspeliensis is an extract of the leaves.

36. The method according to claim 33, wherein the diseases induced by a decrease in the gene and/or protein expression of ECM1 are lichen sclerosus or lipoid proteinosis.

37. The method according to claim 34, wherein the extract is present in the dermatological or pharmaceutical composition in a concentration of from 0.001% to 5% by weight, relative to the total weight of the composition.
